(19) United States
(12) Patent Application Publication
(10) Pub. No.: US 2026/0118334 A1
(43) Pub. Date: Apr. 30, 2026

(54) Electric Vehicle Battery Status Detection System

(71) Applicant: Joyson Safety Systems Acquisition LLC, Auburn Hills, MI (US)

(72) Inventors: Salvatore Brauer, Royal Oak, MI (US); Caleb Breckenridge, Millington, MI (US); Leonard Cech, Brighton, MI (US)

(21) Appl. No.: 19/003,879

(22) Filed: Dec. 27, 2024

Related U.S. Application Data
(60) Provisional application No. 63/616,007, filed on Dec. 29, 2023.
(60) Provisional application No. 63/615,992, filed on Dec. 29, 2023.

(51) Int. Cl. G01N 33/00; B60L 58/18; H01M 10/42

(52) U.S. Cl. CPC G01N 33/0062; G01N 33/0073; H01M 10/4285; B60L 58/18; H01M 2250/20

(57) ABSTRACT

A battery status detection system for an electric vehicle battery pack includes a tube coupled at one end to a valve and at the other end to the battery pack. The system further includes a gas analysis chamber which is coupled to the valve and includes a gas analysis sensor. A pump is coupled to the gas analysis chamber and configured to move gas through the tube, the valve, and the gas analysis chamber. A controller includes a processor which is configured to start the pump, open the valve, and analyze the gas moved by the pump using the gas analysis sensor. By analyzing the composition of the gas, the battery status detection system is able to detect and/or predict dangerous conditions in the battery pack.

### CROSS REFERENCE TO RELATED APPLICATIONS

This application claims the benefit of U.S. Provisional Application No. 63/616,007 filed on Dec. 29, 2023, and claims the benefit of U.S. Provisional Application No. 63/615,992 filed on Dec. 29, 2023, the contents of which are incorporated herein by reference in their entirety.

### TECHNICAL FIELD

The present disclosure relates to safety devices for electric vehicles. In particular, the disclosure relates to a battery status detection system for monitoring battery health in an electric vehicle. Electric vehicles may include any vehicle capable of being powered by an electric propulsion system, such as automobiles, motorcycles, and boats, for example.

### BACKGROUND

Electric vehicles represent a rapidly growing segment of the vehicle market, particularly the automobile market. These vehicles utilize an electric motor propulsion system in lieu of the traditional internal combustion engine (ICE) propulsion system. To power the electric motor, or motors, of an electric vehicle, a large battery pack stores sufficient electrical energy to enable a suitable range (e.g., hundreds of miles before recharging). A common battery cell for use in an electric vehicle battery pack is a lithium-ion battery cell.

It is well understood that lithium-ion battery cells, as well as other battery cells, have several drawbacks. Among them is their ability to catch fire when damaged, for example by an impact or a short circuit. In some cases, there can be little warning which can put people and property in immediate danger. However, with detection equipment, a faulty battery cell could be identified and neutralized before any hazardous condition develops. There is a need for a system which can detect and identify problems within electric vehicle battery packs.

Additionally, these systems could allow for proper preventative maintenance of battery packs.

### SUMMARY

In various implementations, a battery status detection system for an electric vehicle battery pack comprises a tube comprising a first end and a second end. The first end is coupled to a valve and the second end is coupled to the battery pack. A gas analysis chamber comprises a gas analysis sensor and the valve is coupled to the gas analysis chamber. A pump is coupled to the gas analysis chamber and is configured to move gas through the tube, the valve, and the gas analysis chamber. The battery status detection system also comprises a controller comprising a processor and a memory, wherein the processor executes instructions stored in the memory causing the processor to start the pump, open the valve, and analyze the gas moved by the pump using the gas analysis sensor.

In some implementations, a battery status detection system for an electric vehicle battery pack comprises a plurality of tubes each comprising a first end and a second end. Each of the first ends are coupled to a valve and each of the second ends are coupled to each of a plurality of battery modules of the battery pack. The valve is capable of being opened or closed with respect to each of the plurality of tubes. A gas analysis chamber comprises a gas analysis sensor and the valve is coupled to the gas analysis chamber. A pump is coupled to the gas analysis chamber and configured to move gas through the plurality of tubes, the valve, and the gas analysis chamber. The battery status detection system also comprises a controller comprising a processor and a memory, wherein the processor executes instructions stored in the memory causing the processor to start the pump, open the valve with respect to one of the plurality of tubes and close the valve with respect to each of the remaining tubes of the plurality of tubes, and analyze the gas moved by the pump using the gas analysis sensor.

In other implementations, a battery status detection system for an electric vehicle battery pack comprises a plurality of first tubes each comprising a first end and a second end. Each of the first ends are coupled to a first valve and each of the second ends are coupled to each of a first plurality of battery cells of the battery pack. The first valve is capable of being opened or closed with respect to each of the plurality of first tubes. A plurality of second tubes each comprise a first end and a second end. Each of the first ends are coupled to a second valve and each of the second ends are coupled to each of a second plurality of battery cells of the battery pack. The second valve is capable of being opened or closed with respect to each of the plurality of second tubes. A third valve is coupled to the first valve and the second valve and is capable of being opened or closed with respect to each of the first valve and the second valve. A gas analysis chamber comprises a gas analysis sensor and the third valve is coupled to the gas analysis chamber. A pump is coupled to the gas analysis chamber and configured to move gas through the plurality of first tubes, the plurality of second tubes, the first valve, the second valve, the third valve, and the gas analysis chamber. The battery status detection system also comprises a controller comprising a processor and a memory, wherein the processor executes instructions stored in the memory causing the processor to start the pump, open the first valve with respect to one of the plurality of first tubes and close the first valve with respect to each of the remaining tubes of the plurality of first tubes, open the third valve with respect to the first valve and close the third valve with respect to the second valve, and analyze the gas moved by the pump using the gas analysis sensor.

### BRIEF DESCRIPTION OF THE DRAWINGS

The drawings are merely exemplary to illustrate steps, structure, and certain features that can be used singularly or in combination with other features. The disclosure should not be limited to the implementations shown.

FIG. 1 is a perspective view of a battery pack.

FIG. 2A is a cross sectional view of the battery pack of FIG. 1 along the line A-A, showing a plurality of battery modules within the battery pack.

FIG. 2B is a perspective view of one of the plurality of battery modules of FIG. 2A with a cutout portion showing a plurality of battery cells within the battery module.

FIG. 3 is a system view of a first implementation of a battery status detection system.

FIG. 4 is a system view of a second implementation of the battery status detection system.

FIG. 5 is a system view of a third implementation of the battery status detection system.

FIG. 6 is a perspective view of a single battery cell from FIG. 5.

### DETAILED DESCRIPTION

The present disclosure relates to safety devices for electric vehicles. The devices, assemblies, and methods disclosed herein provide for a battery status detection system for an electric vehicle battery pack. The battery status detection system moves gas through the system and detects the composition of that gas using a gas analysis sensor. A controller comprising a processor and a memory compares the composition of the gas to predefined thresholds set for specific constituents in order to determine the health of the battery pack. When specific constituents in the gas are detected above their thresholds, the battery status detection system can alert the vehicle operator that an unsafe condition exists and that certain safety measures should be taken.

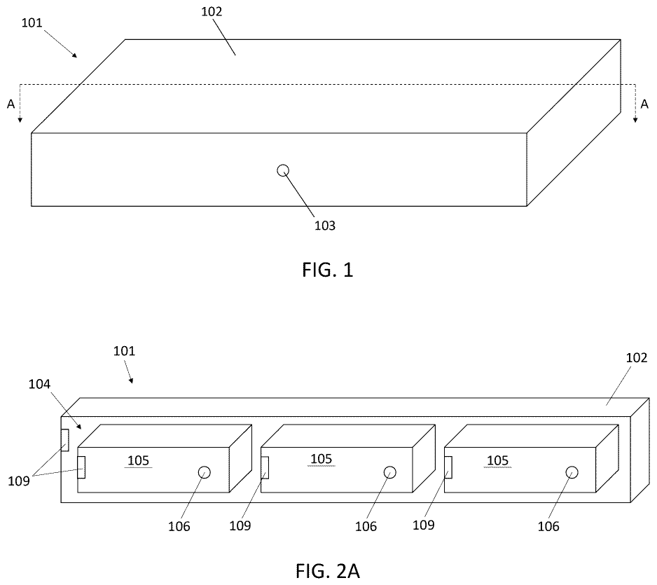

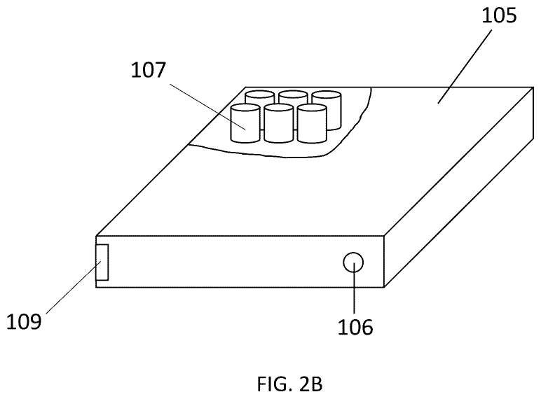

As shown in FIGS. 1-2B, a battery pack 101 comprises a housing 102 which defines a housing communication chamber 104. At least one battery module 105 is disposed within the housing communication chamber 104. Disposed within each battery module 105 is a plurality of battery cells 107. A plurality of environmental sensors 109 may be disposed within the housing communication chamber 104 and/or on/within/adjacent to each battery module 105.

The environmental sensors 109 may be chosen from temperature sensors, pressure/force sensors, humidity sensors, or others.

The battery modules 105 and the battery cells 107 may be electrically wired together to provide a desirable energy storage capacity and power output. Typical battery cells in electric vehicles include lithium-ion, lead-acid, nickel-cadmium, and nickel-metal hydride, for example, with lithium-ion being particularly popular. Unfortunately, lithium-ion battery cells, for example, may suffer from thermal breakdown resulting from electrical shorts and other causes, such as high force impacts. When this happens, chemical reactions within the battery cells may produce gases such as methane, ethylene, carbon dioxide, carbon monoxide, dimethyl ether, ethylene carbonate, diethyl carbonate, ethyl methyl carbonate, hydrogen, and other volatile organic compounds. If detected before failure of the battery cell (e.g., fire), electric vehicle battery packs can be serviced and may be able to remain in operation.

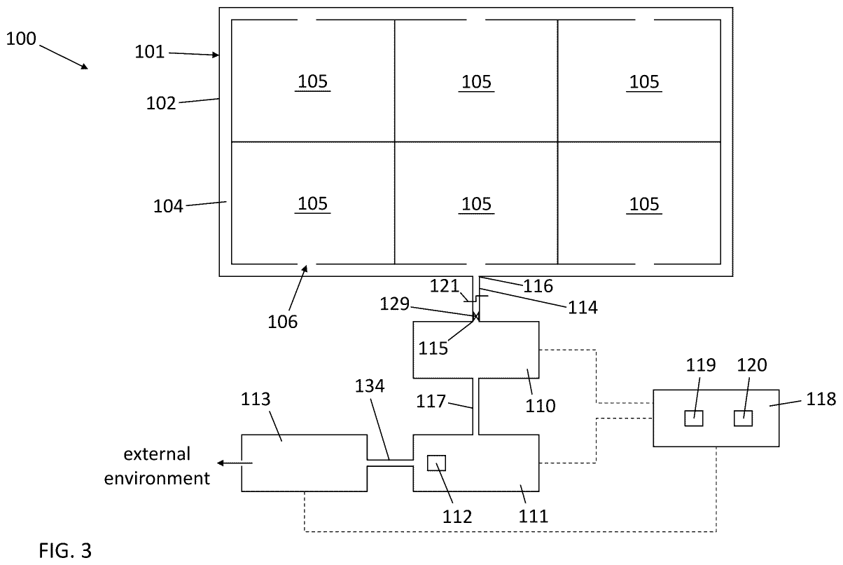

Referring now to FIG. 3, a first implementation of a battery status detection system 100 comprises a battery pack 101 comprising a housing 102 which defines a housing communication chamber 104 with a plurality of battery modules 105 disposed therein. In this example, six battery modules 105 are disposed within the housing communication chamber 104, however any number of battery modules 105 may be used, depending on considerations such as available space and required energy storage capacity. Each battery module 105 defines a module communication port 106 that is in fluid communication with the housing communication chamber 104. Each module communication port 106 may be, for example, an open hole or a one-way check valve, passively operable by pressure, to enable gas to leave, but not enter, the battery module 105.

The battery pack 101 is coupled to a tube 114 comprising a first end 115 and a second end 116. The second end 116 is coupled to the battery pack 101 via a housing communication port 103 (see FIG. 1) defined by the housing 102 which provides fluid communication between the housing communication chamber 104 and the tube 114. The housing communication port 103 may be, for example, an open hole or a one-way check valve passively operable by pressure to enable gas to leave, but not enter, the housing communication chamber 104. The first end 115 is coupled to a valve 110. Optionally, a one-way check valve 121, passively operable by pressure, may be disposed anywhere within the tube 114 to ensure gas only flows from the battery pack 101 to the valve 110 and not vice versa. The tube 114 may be made from any material as required by vehicle characteristics. For example, the tube 114 may comprise PVC or other rigid plastic tubing, PEX or other flexible plastic tubing, or rigid or flexible metal tubing.

The length of the tube 114 and the routing of the tube 114 throughout the vehicle may vary depending on vehicle characteristics. For example, the valve 110 and the rest of the battery status detection system 100 (described below) may be located any distance from the battery pack 101 as required.

The valve 110 comprises a solenoid 129 located adjacent the first end 115 of the tube 114 and is integrally part of the valve 110. The valve 110 is coupled to a gas analysis chamber 111 via a conduit 117. The gas analysis chamber 111 comprises a gas analysis sensor 112 and is further coupled to a pump 113 via a conduit 134. The pump 113 may be a fan, a vacuum, or any other device capable of pulling gas through the battery status detection system 100 by producing a negative pressure thereby pulling gas from the battery pack 101 through to the gas analysis chamber 111 and ultimately out to an external environment (e.g., vehicle cabin or external of the vehicle). The conduits 117, 134 may be similar to tube 114, or the valve 110, gas analysis chamber 111, and pump 113 may all be integrated into one common housing or other structure, so long as fluid communication is possible between the components. The gas analysis chamber 111 may be defined by a housing or other structure, such as a plastic or metal housing, that allows gas to flow over or through the gas analysis sensor 112.

One representative gas analysis sensor 112 is based on transmission spectroscopy using a broad spectral band light source which emits light into the gas analysis chamber 111. An example of a transmission spectrometer sensor is a Michelson interferometer (other spectrometers could also be used, such as a Fabry-Perot or grating spectrometer). The Michelson interferometer comprises an optical system located adjacent to or spaced apart from the broad spectral band light source. The Michelson interferometer further comprises a fixed mirror, a movable mirror, and a partially reflective mirror. By controlling the movable mirror as a function of time, the system sweeps through all individual wavelengths within the broad spectral band light source, typically comprising visible, near infrared, and mid-infrared wavelengths. A digital detector, which detects the received light at each wavelength, produces a voltage versus time signal which is then processed through a digital Fourier transform after each full mirror sweep to produce an absorbance spectrum (i.e., amplitude versus wavelength).

Since each chemical molecule has a unique deterministic absorbance spectrum, digital algorithms (e.g., principal least squares, principal components analysis, chemometrics, AI, etc.) can be used to determine the chemicals within the sample and estimate their concentrations within the sample. In other implementations, other gas analysis sensors could be used. For example, electrochemical MEMs sensors operate by having unique cathode/anode chemistries for multiple target chemicals that generate a deterministic voltage as a function of the concentration in a gas sample.

The valve 110 may be an electromechanical solenoid valve as known in the art and used in numerous industries including industrial, medical, and automotive for gas and/or fluid control. In some implementations, one or more discrete solenoid microvalves or a micro-manifold (several valves integrated into a single component) are used. For example, the valve 110 may be made by companies such as miniValve (https://minivalve.com) or the Lee Company (https://www.theleeco.com). Such valves are connected to input and output ports (e.g., connected to airtight tubes) and are electromagnetically controllable to open/close quickly and securely over potentially millions of cycles. A general method to open/close such valves includes providing a positive voltage/current to an electrical coil of a solenoid which moves a plunger to the open or closed position as long as the voltage/current is applied. Such solenoid plungers may also include a mechanical resistance system (e.g., a spring) to assure that the plunger moves back to the correct mechanical state after the voltage/current is turned off. Other components can also be integrated into such valves, for example filters (e.g., particulates, humidity, or chemical specific) and/or one-way check valves depending on the requirements of the battery status detection system 100.

A controller 118 is electronically coupled to the valve 110, gas analysis sensor 112, and pump 113 in order to control the operation of the battery status detection system 100 (electrical coupling represented by dashed lines in FIG. 3, for example). The controller 118 may be directly coupled via wired connections or wirelessly connected through a wireless protocol, such as WIFI or BLUETOOTH. The controller 118 comprises a processor 119 and a memory 120. The processor 119 executes instructions stored on the memory 120 to open the valve 110 (e.g., open/activate the solenoid 129), operate the gas analysis sensor 112, and turn on the pump 113. Therefore, when the battery status detection system 100 is operating, the pump 113 pulls gas out of the plurality of battery modules 105 into the housing communication chamber 104, then through the tube 114, the valve 110, the conduit 117, the gas analysis chamber 111, the conduit 134, the pump 113, and out into the external environment. During this time, the gas analysis sensor 112 may continually or intermittently (e.g., every few seconds or minutes) analyze the gas for its composition.

The memory 120 may also store predefined threshold limits for gas concentration of various gas constituents (discussed above) so that the controller 118 may identify when a hazardous condition exists within the battery pack 101. The controller 118 may continuously operate the battery status detection system 100 or it may operate the system 100 intermittently. For example, the controller 118 may start the pump 113, open the valve 110, and analyze gas moved by the pump 113 using the gas analysis sensor 112 for a period of one minute out of every ten minutes. When not operating, the battery status detection system 100 may sit idle with no gas moving through the system 100.

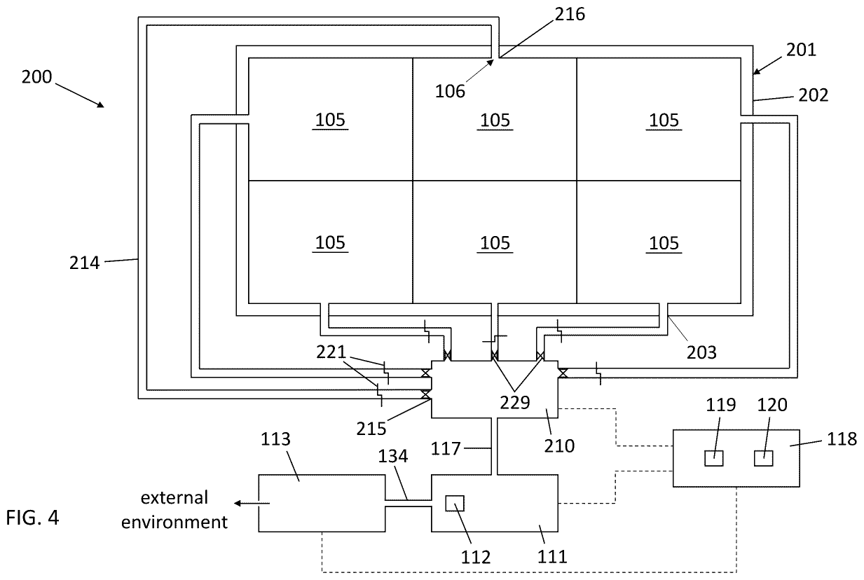

Referring now to FIG. 4, a second implementation of a battery status detection system 200 comprises a battery pack 201 comprising a housing 202 with a plurality of battery modules 105 disposed therein. The system 200 is similar to the system 100, however in this example a valve 210 comprises multiple connections to a plurality of tubes 214, each of the plurality of tubes 214 comprising a first end 215 coupled to the valve 210 and a second end 216 coupled to one of the plurality of battery modules 105. Each of the plurality of tubes 214 may comprise a one-way check valve 221, passively operable by pressure, disposed anywhere within the tubes 214 to ensure gas only flows from the plurality of battery modules 105 to the valve 210 and not vice versa. Similarly, the valve 210 comprises a plurality of solenoids 229, each operable with respect to one of the plurality of tubes 214. This allows the valve 210 to selectively open only one of the plurality of tubes 214 at any given time, therefore allowing the battery status detection system 200 to individually diagnose the status of each of the plurality of battery modules 105. This selectiveness allows the vehicle owner, or manufacturer/others, to service the individual battery modules 105 more efficiently.

As an example, the controller 118 may instruct the valve 210 to open with respect to one of the plurality of tubes 214 and close with respect to each of the remaining tubes 214 of the plurality of tubes 214 (e.g., open one solenoid 229 and close all others). The controller 118 can then instruct the pump 113 to begin pulling gas through the system and instruct the gas analysis sensor 112 to begin analyzing the composition of the gas, similar to the above description for the battery status detection system 100. In this way, the gas analysis sensor 112 will only be analyzing gas that is associated with a single battery module 105 at any given time, rather than the entire battery pack 201, therefore allowing the controller 118 to identify an individual battery module 105 in need of service.

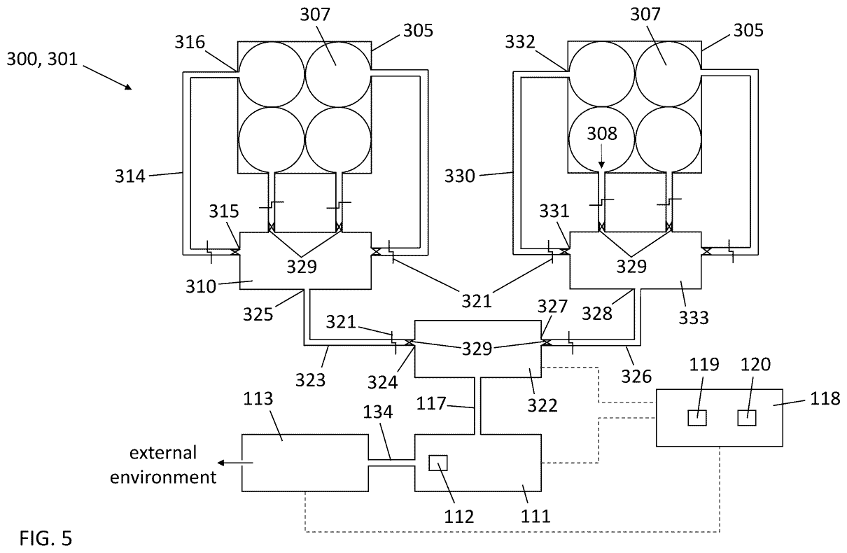

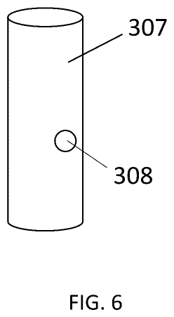

Referring now to FIGS. 5-6, a third implementation of a battery status detection system 300 allows for even further granularity in diagnosing problems within a plurality of battery modules 305. Each battery module 305, as discussed above, may contain dozens or even hundreds of battery cells 307. Each battery cell 307 may comprise a cell communication port 308. Each cell communication port 308 may be, for example, an open hole or a one-way check valve, passively operable by pressure, to enable gas to leave, but not enter, the battery cell 307.

For clarity, FIG. 5 shows two battery modules 305 comprising four battery cells 307 each.

However, the system 300 may be scaled to any number of battery modules 305 and battery cells 307 as needed.

The battery status detection system 300 comprises a plurality of first tubes 314, each comprising a first end 315 and a second end 316, each of the first ends 315 coupled to a first valve 310 and each of the second ends 316 coupled to a cell communication port 308 of each of a first plurality of battery cells 307. The system 300 further comprises a plurality of second tubes 330, each comprising a first end 331 and a second end 332, each of the first ends 331 coupled to a second valve 333 and each of the second ends 332 coupled to a cell communication port 308 of each of a second plurality of battery cells 307. Therefore, the system 300 can evaluate the health of each individual battery cell 307 of a battery pack 301.

To further accomplish this goal, a third valve 322 is coupled to each of the first valve 310 and the second valve 333 by a third tube 323 and a fourth tube 326, respectively. A first end 324 of the third tube 323 is coupled to the third valve 322 while a second end 325 of the third tube 323 is coupled to the first valve 310. Similarly, a first end 327 of the fourth tube 326 is coupled to the third valve 322 while a second end 328 of the fourth tube 326 is coupled to the second valve 333. Similar to the implementations described above, the third valve 322 incorporates solenoids 329 to open and close access to the third tube 323 and the fourth tube 326, and each of the third tube 323 and the fourth tube 326 may comprise a one-way check valve 321.

As an example of the operation of the battery status detection system 300, the controller 118 may instruct the third valve 322 to open with respect to the first valve 310 and close with respect to the second valve 333 (e.g., open one solenoid 329 and close the other). Additionally, the controller 118 may then instruct the first valve 310 to open with respect to one of the plurality of first tubes 314 and close with respect to each of the remaining tubes of the plurality of first tubes 314 (e.g., open one solenoid 329 and close all others). The controller 118 can then instruct the pump 113 to begin pulling gas through the system and instruct the gas analysis sensor 112 to begin analyzing the composition of the gas, similar to the above description for the battery status detection system 100. In this way, the gas analysis sensor 112 will only be analyzing gas that is associated with a single battery cell 307 at any given time, therefore allowing the controller 118 to identify an individual battery cell 307 in need or service.

What is claimed is:

1. A battery status detection system for an electric vehicle battery pack comprising:
a tube comprising a first end and a second end, the first end coupled to a valve and the second end coupled to the battery pack;
a gas analysis chamber comprising a gas analysis sensor, wherein the valve is coupled to the gas analysis chamber;
a pump coupled to the gas analysis chamber and configured to move gas through the tube, the valve, and the gas analysis chamber; and
a controller comprising a processor and a memory;
wherein the processor executes instructions stored in the memory, the instructions causing the processor to start the pump, open the valve, and analyze the gas moved by the pump using the gas analysis sensor.

2. The battery status detection system of claim 1, wherein the battery pack comprises a plurality of battery modules.

3. The battery status detection system of claim 2, wherein the battery pack defines a communication chamber and each of the plurality of battery modules define a communication port, wherein the communication chamber is in fluid communication with all of the communication ports of the plurality of battery modules.

4. The battery status detection system of claim 3, wherein the second end of the tube is coupled to the communication chamber.

5. The battery status detection system of claim 4, wherein the pump is further configured to move gas through the communication chamber.

6. The battery status detection system of claim 3, wherein the battery pack further comprises an environmental sensor.

7. The battery status detection system of claim 6, wherein the environmental sensor is disposed within the communication chamber.

8. The battery status detection system of claim 6, wherein the environmental sensor comprises a plurality of environmental sensors, wherein each of the plurality of environmental sensors is disposed on or within each of the plurality of battery modules.

9. The battery status detection system of claim 1, wherein the tube is closable by a one-way check valve disposed within the tube.

10. The battery status detection system of claim 9, wherein the one-way check valve is passively operated by pressure.

11. The battery status detection system of claim 1, wherein opening the valve comprises the instructions causing the valve to activate a solenoid.

12. A battery status detection system for an electric vehicle battery pack comprising:
a plurality of tubes each comprising a first end and a second end, each of the first ends coupled to a valve and each of the second ends coupled to each of a plurality of battery modules of the battery pack, the valve capable of being opened or closed with respect to each of the plurality of tubes;
a gas analysis chamber comprising a gas analysis sensor, wherein the valve is coupled to the gas analysis chamber;
a pump coupled to the gas analysis chamber and configured to move gas through the plurality of tubes, the valve, and the gas analysis chamber; and
a controller comprising a processor and a memory;
wherein the processor executes instructions stored in the memory, the instructions causing the processor to start the pump, open the valve with respect to one of the plurality of tubes and close the valve with respect to each of the remaining tubes of the plurality of tubes, and analyze the gas moved by the pump using the gas analysis sensor.

13. The battery status detection system of claim 12, wherein the battery pack further comprises an environmental sensor.

14. The battery status detection system of claim 13, wherein the environmental sensor comprises a plurality of environmental sensors, wherein each of the plurality of environmental sensors is disposed on or within each of the plurality of battery modules.

15. The battery status detection system of claim 12, wherein each of the plurality of tubes is closable by a one-way check valve disposed within each of the plurality of tubes.

16. The battery status detection system of claim 15, wherein the one-way check valve disposed within each of the plurality of tubes is passively operated by pressure.

17. The battery status detection system of claim 12, wherein opening the valve with respect to one of the plurality of tubes and closing the valve with respect to each of the remaining tubes of the plurality of tubes comprises the instructions causing the valve to activate a plurality of solenoids.

18. A battery status detection system for an electric vehicle battery pack comprising:
a plurality of first tubes each comprising a first end and a second end, each of the first ends coupled to a first valve and each of the second ends coupled to each of a first plurality of battery cells of the battery pack, the first valve capable of being opened or closed with respect to each of the plurality of first tubes;
a plurality of second tubes each comprising a first end and a second end, each of the first ends coupled to a second valve and each of the second ends coupled to each of a second plurality of battery cells of the battery pack, the second valve capable of being opened or closed with respect to each of the plurality of second tubes;
a third valve, wherein the first valve and the second valve are coupled to the third valve, the third valve capable of being opened or closed with respect to each of the first valve and the second valve;
a gas analysis chamber comprising a gas analysis sensor, wherein the third valve is coupled to the gas analysis chamber;
a pump coupled to the gas analysis chamber and configured to move gas through the plurality of first tubes, the plurality of second tubes, the first valve, the second valve, the third valve, and the gas analysis chamber; and
a controller comprising a processor and a memory;
wherein the processor executes instructions stored in the memory, the instructions causing the processor to start the pump, open the first valve with respect to one of the plurality of first tubes and close the first valve with respect to each of the remaining tubes of the plurality of first tubes, open the third valve with respect to the first valve and close the third valve with respect to the second valve, and analyze the gas moved by the pump using the gas analysis sensor.

19. The battery status detection system of claim 18, wherein the battery pack further comprises an environmental sensor.

20. The battery status detection system of claim 19, wherein the battery pack further comprises a first battery module and a second battery module, the first plurality of battery cells and the second plurality of battery cells being disposed within the first battery module and the second battery module, respectively.

21. The battery status detection system of claim 20, wherein the environmental sensor comprises two environmental sensors, wherein each of the two environmental sensors is disposed on or within the first battery module and the second battery module, respectively.

22. The battery status detection system of claim 18, wherein each of the plurality of first tubes and each of the plurality of second tubes are closable by a plurality of one-way check valves, wherein one of the plurality of one-way check valves is disposed within each of the plurality of first tubes and each of the plurality of second tubes.

23. The battery status detection system of claim 22, wherein the plurality of one-way check valves are passively operated by pressure.

24. The battery status detection system of claim 18, wherein opening the first valve with respect to one of the plurality of first tubes and closing the first valve with respect to each of the remaining tubes of the plurality of first tubes comprises the instructions causing the first valve to activate a first plurality of solenoids.

25. The battery status detection system of claim 18, wherein the first valve is coupled to the third valve by a third tube comprising a first end coupled to the third valve and a second end coupled to the first valve, and the second valve is coupled to the third valve by a fourth tube comprising a first end coupled to the third valve and a second end coupled to the second valve, wherein the third tube and the fourth tube are each closable by a one-way check valve disposed within the third tube and the fourth tube.

26. The battery status detection system of claim 25, wherein the one-way check valves disposed in the third tube and the fourth tube are passively operated by pressure.

27. The battery status detection system of claim 24, wherein opening the third valve with respect to the first valve and closing the third valve with respect to the second valve comprises the instructions causing the third valve to activate a second plurality of solenoids.
